(19) United States
(12) Patent Application Publication
(10) Pub. No.: US 2002/0165233 A1
(43) Pub. Date: Nov. 7, 2002

(54) Quinoxaline containing medicaments for post exposure prophylaxis of an HIV infection

(76) Inventors: Kazuyasu Mori, Tsukuba (JP); Yasuhiro Yasutomi, Mie (JP); Kazushiqe Sugama, Nara (JP); Shuzo Sawada, Kita-ku (JP); Helga Rubsamen-Waigmann, Bad Soden/Taunus (DE)

Correspondence Address: NATH & ASSOCIATES, 1030 15th STREET, 6TH FLOOR, WASHINGTON, DC, 20005, US

(21) Appl. No.: 09/988,798

(22) Filed: Nov. 20, 2001

Related U.S. Application Data
(63) Continuation of application No. PCT/EP00/04646, filed on May 22, 2000.

(30) Foreign Application Priority Data
May 21, 1999 (GB) 9911887.9

(51) Int. Cl. A61K 31/498

(52) U.S. Cl. 514/248; 514/249

(57) ABSTRACT

Disclosed is the use of an antiviral substance which, when given alone for 14 days, shows a mean initial suppression of viral load by 1.4 log or more and which does not reduce the number of lymphocytes, granulocytes and macrophages as determined by differential blood count after 12 weeks of treatment for the manufacture of a medicament for the immune system-assisted post-exposure prophylaxis of an HIV infection.

### FIELD OF THE INVENTION

The present invention relates to a method for the prophylactic treatment of a (presumed) infection with Human Immunodeficiency Virus (HIV) to prevent, alleviate or at least substantially delay diseases or conditions which are caused, mediated or aggravated by the virus. The invention further relates to the use of certain antiviral substances in the preparation of medicaments for use in such a method.

### BACKGROUND OF THE INVENTION

For the treatment of HIV-infections, 3 classes of drugs are currently available, namely (a) nucleosidic drugs which inhibit the viral reverse transcriptase (RT) as analogs of its substrate (NRTI), (b) non-nucleosidic compounds which inhibit the same enzyme by binding to a hydrophobic pocket of the RT (NNRTI) and (c) inhibitors of the viral protease. Both monotherapies and combination therapies using certain representatives of these classes have been tried in the past to treat or prevent an HIV infection. An established HIV infection, as used herein, is present when antibodies against HIV can be detected in the subject. At the current state of analytical techniques (May 1999) it is not yet routinely possible to detect HIV in a patient directly after exposure. The virus is obviously there but in most cases at a concentration too low to allow for its detection.

Attempts to combat HIV may be classified in (a) therapies to control an established infection (antibodies against HIV detectable) and (b) prophylactic treatments (antibodies against HIV not yet detectable but exposure to HIV proven or likely) with the aim to prevent an infection. The present invention relates to the latter situation.

Current State in HIV Therapy

Historically, HIV therapy first involved treatment of individuals with an established HIV infection, most of them already showing signs of immunodeficiency, with one RT Inhibitor only (monotherapy). However, due to the unfortunate fact that HIV rapidly shows mutations and thus invokes drug resistance when treated with a single substance, the current state of the art in the treatment of HIV-1 infections involves the administration of a combination of at least two, more commonly three substances (triple therapy). Such a combination usually involves at least one nucleosidic RT inhibitor (most commonly lamivudine, and often a combination of lamivudine and zidovudine) and a protease inhibitor or a NNRTI. The triple therapy has been very successful in suppressing HIV and progression to AIDS was remarkably reduced. However, a complete eradication of HIV by this therapy has not been possible and reappearance of high viral loads has increasingly been observed due to the generation of multi-resistant viruses.

The question of the best point in time to start an HIV treatment is presently under debate. Most experts advise that one should rather abstain from or delay treatment of asymptomatic HIV-infected (seroconverted) patients as long as these still have high CD4 cell numbers (above 500 CD4 cells/mm³) and low numbers of copies of HIV RNA (<10,000-20,000 copies/ml). This is inter alia expressed in the German-Austrian Guidelines for Antiretroviral Therapy of HIV Infection of December 1997, published in European Journal of Medical Research, Dec. 31, 1997, p. 534-541 and incorporated herein by reference.

The following is a quotation from these Guidelines which are jointly issued by the Deutsche AIDS Gesellschaft, the Österreichische AIDS Gesellschaft, the Robert-Koch-Institute and various other organizations:

“While theoretically, antiviral treatment of HIV infection should be started as early as possible, previous trials with agents less active than those available today have not demonstrated any clinical benefit for asymptomatic patients with high CD4 cell counts. Starting antiretroviral treatment means strict adherence to a medication regimen for an unknown time and may have profound and negative consequences for the quality of life. Due to cross-resistance of the available agents, only two three-drug regimens can be administered sequentially at the present time without incomplete (i.e., for at least one agent) resistance. Thus, early treatment may actually compromise therapeutic options for the future.”

Further evidence for the above is provided by the fact that current standard NRTIs such as zidovudine and lamivudine are only approved for the treatment of a progressive immunosuppression or for patients undercutting a lower threshold of CD4 cell counts.

However, attempts directed to an early treatment of a HIV infection have also been reported. The group of Bruce Walker [Rosenberg et al, Science 1997, 1447-1450] treated patients with an acute HIV-1 retroviral syndrome using a three-drug therapy. An acute retroviral syndrome is characterized by constitutional symptoms, malaise, generalized lymphadenopathy, and a morbilliform rash. High levels of HIV RNA are initially found in these patients. The treatment reported by Rosenberg et al was started after the patients were found to be HIV-1-RNA positive by the Amplicor HIV Monitor Test (Roche) and negative in a HIV-1/2 enzyme immunoassay provided by Abott Laboratories, i.e. at an early stage of clinically manifest infection with proof of presence of virus and before seroconversion. They found that these patients developed a vigorous proliferative HIV-1-specific CD4⁺ T cell response similar to the response observed in individuals with longterm nonprogressive infection. They further observed that the gradual generation of an HIV antigen (p24)-specific proliferative response is associated with the lowering of HIV-1 viral load during primary HIV-1 infection and suggest that early, aggressive treatment of primary infection may facilitate the generation of these responses. Moreover, Rosenberg et al. hypothesized that these responses contribute to immunological control of virus replication although disease progression in HIV-1 infection was said to be likely to depend on several factors. It seems that Rosenberg et al. did not believe that this proliferative response alone could be sufficient to control the virus. At the end of their paper, they suggested to test methods for the induction of virus-specific CD4⁺ T cells in chronically infected individuals (i.e. not in primary infection), in conjunction with control of viral load by antiviral drugs. Such induction could possibly be achieved with DNA vaccination.

According to McMichael (Cell, Vol. 93, 673-676, May 29, 1998), a central reason for the failure of the immune response to adequately control HIV must be that the CD4⁺ T helper cells, that respond to the virus, are at the same time its targets. T helper cell response to HIV has always been hard to investigate because of difficulties in generating any response at all in vitro. Referring to the work of Rosenberg et al (see above) McMichael notes that CD4⁺ T cells responding to the HIV Gag antigen could be identified in patients treated very early after infection and formulated the hypothesis that inhibition of virus replication by the drug rescued the T helper cell response. This would imply, according to McMichael, that the HIV-specific T helper cell response starts normally but, in the absence of drug treatment, is aborted by infection of the responding T cells. The result is, in effect, a state of T helper cell tolerance to HIV by deletion of the HIV specific CD4⁺ helper cell clones.

Very recently, the group of Walker [Abstract of 6th Conference on Retrovirus and Opportunistic Infections, Jan. 31-Feb. 4, 1999] has found immune control of HIV after suspension of therapy in one patient who was treated after infection but before complete seroconversion with a three-drug therapy comprising inter alia hydroxyurea, a compound which is is known to invoke lymphopenias, for 176 days. This patient showed no viremia rebound for 551 days following discontinuation of treatment. Vigorous HIV specific T-helper and CD8 cytotoxic T-lymphocytes repsonses provided evidence of a T-cell mediated immune control of HIV after discontinuation of therapy.

Current State in HIV Prophylaxis

Considerable efforts in public education have been made to prevent HIV infections. However, these efforts have not always been successful, and HIV exposures by unprotected sexual contact still occur today, In addition, occupational transmission of HIV may result from e.g. needle-stick and cut injuries, contact with an open wound or non-intact skin, or from exposure of mucous membranes. A further source for HIV transmission may be the use of HIV-contaminated injection equipment which is a particular problem with drug-addicted persons. Thus, there still is a need for an effective and reliable medical prophylaxis against HIV.

Prior art attempts to obtain and maintain effective protection against HIV-1 by prophylactic drug administration have been reported but have not always been successful.

For example, Schleif et al. (AIDS Research and Human Retroviruses, Vol. 10, No. 1, 1994) attempted the prophylaxis of a HIV-1 Infection in Chimpanzees with the NNRTI pyridinone derivative L-696,229 (Merck). The virus challenge was performed 1 hr following a dose of the inhibitor, i.e. the inhibitor was given pre-exposure and the treatment was continued for 20-30 weeks post challenge. It was found that the treated animals developed delayed infections but the treatment was not fully efficient and did not provide sufficient protection against infection and later viremia.

On the other hand, in animal models employing non-human retroviruses Tsai et al (Science (1995), Vol. 270, 1197-1199) have reported that an SIV infection of macaques could be prevented by administration of PMPA 48 h before, 4 h after and 24 h after virus inoculation for 4 weeks, observing the monkeys for 56 weeks. Since full protection of the monkey was achieved, this study does not allow for conclusions on whether PEP would modify the course of the disease, and an immunological analysis was not carried out.

Likewise, Ruprecht et al (J. Acquired Immune Deficiency Syndromes 3:591-600, (1990)) demonstrated in the Rauscher Leukemia Virus System that a combination of AZT and gamma Human Interferon a after retroviral exposure prevented viremia and disease and protected the animals from challenge with a wild type virus. They conclude (p. 598) that their experiment is a variation of classical vaccination, However, since lentiviruses (a subgroup of retroviruses) contain additional regulatory genes like tat, rev or nef, the authors caution (p. 592) that results obtained in this type C animal model may not be generally extrapolated to HIV-infected humans, because of species differences in drug metabolism.

Furthermore, Rosenwirth et al (38th ICAAC, Sep. 24-27, 1998) demonstrated that post exposure prophylaxis (PEP) with PMPA in RT-SHIV infected rhesus monkeys had a vaccination effect protecting the animals against wild-type challenge similar to the vaccination by attenuated SIV reported by the group of Derosiers, However, no implications on PEP for preventing diseases can be derived.

Summarizing the above, it can be stated that no well-defined and generally applicable procedure for PEP exists to date which would prevent infection and/or disease in all HIV-exposed humans.

In practice, AZT (zidovudine) has widely been used for post-exposure administration in man since 1989 and often provided good results. A case-control study has shown that prophylaxis with only zidovudine probably has an 80% protective effect. This is summarized in the German-Austrian Recommendations for HIV Postexposure Prophylaxis, published in European Journal of Medical Research, Oct. 14, 1998, p. 485-498, and incorporated herein by reference.

These guidelines advocate a standard HIV Postexposure Prophylaxis (PEP) using a combination of two reverse transcriptase inhibitors and one protease inhibitor, which combination should be more aggressive against HIV than zidovudine alone and reduce the risk of drug resistances. In view of the successes obtained with zidovudine in the past, one component of this combination is recommended to be zidovudine, provided that the source person of the infection has not developed resistance to it.

The guidelines recommend starting the treatment as soon as possible after exposure to attain the maximum protective effect. However, it is stated that beginning the treatment later than 72 h after exposure would in any case be senseless. Ideally, the prophylaxis should be begun within the first 2 h after exposure. While no scientific rationale for these recommendations is explicitly given in the guidelines, it is fair to assume that the purpose of the recommended prophylactic measures simply was to reduce the number of virus as much as possible at a very early time in order to prevent infection. Reference is made (chapter 1.3) to experimental studies revealing that the time span between uptake of HIV until attachment to the host cell amounts to up to 2 hours, until the first transcription of viral RNA, to 12 hours, and until the first formation of viral particles, another 12 hours. It should be easier to attack HIV before it is attached and incorporated by a host cell, which in some cases may not be well accessible by blood circulation. Implicitly, these guidelines assume that early treatment is useless once prevention of infection can no longer be assumed to be likely, i.e. after 72 h following exposure.

It would of course be ideal to eliminate the virus completely by any kind of therapy. However, past experiences have shown that this goal is very difficult to achieve, since a reservoir of replication-competent HIV is established soon after infection and persists even after years of aggressive antiretroviral treatment. Therefore conventional antiretroviral therapy cannot be discontinued without the risk of a HIV rebound. This in turn aggravates the problem of the side-effects of the conventional HIV therapy.

To overcome this drawback, several groups have tried to stimulate the immune system of the treated host by vaccination. However, it turned out that these attempts were not really successful (see e.g. Linhart et al., AIDS Research and Human Retroviruses 13 (7) (1997): 593-599). As of today, there is no vaccine approved for HIV treatment.

### SUMMARY OF PRIOR ART AND OBJECT OF THE INVENTION

In view of the above, the current state of the art in combatting HIV may be summarized as follows:

(a) An strong reduction of the viral load after established infection (seroconversion) is considered as desirable. The best time to start therapy, however, is still under debate. Means to achieve viral load reduction may be an aggressive triple combination therapy or highly active antiretroviral therapy.

(b) However, in view of the concerns relating to resistance with the current antiviral therapies and their complicated use there has been a reluctance to start a therapy immediately after a patient has been shown to be HIV seropositive, as long as he/she still has high counts of CD4 cells and low counts of viral RNA.

(c) Immunostimulation by vaccination after seroconversion has been tried but was not really successful.

(d) Very early post exposure prophylactic administration of zidovudine before seroconversion has been tried and shown success in avoiding infection in a considerable number of patients, although not in all. This medication was based on the paradigm that viral load should be reduced as much as possible at a time when the virus still is particularly vulnerable, with the aim to prevent infection. The current state of the art is to give a three-drug combination (containing zidovudine) for post exposure prophylaxis rather than zidovudine alone. It seems that sufficient comparative data is not yet available.

(e) Starting PEP more than 72 h post exposure is not considered useful.

(f) The group of Walker has reported that one patient who was treated before complete seroconversion with a three-drug therapy containing hydroxyurea for 176 days showed no viremia rebound for 551 days following discontinuation of treatment. In this patient, evidences of T-cell mediated immune control of HIV after discontinuation of therapy were found.

In view of the above, the present invention inter alia aims at providing the necessary criteria (in terms of suitable substances and the timing of the administration thereof) how to achieve an excellent and reliable HIV prophylaxis which is useful at least in the majority of exposures. The invention particularly strives for attaining such a prophylaxis-without unduly compromising the long-term therapeutic outcome. This is achieved by optimizing the primary immunological control of the infection by the HIV-exposed person while avoiding or at least minimizing immunological side-effects. A further object is to define a suitable therapeutic regimen for such a prophylaxis. Moreover, the invention aims at providing medicaments for use in the prophylaxis of an HIV infection and an emergency pharmaceutical preparation kit to be applied following a suspected HIV exposure.

### SUMMARY OF THE INVENTION

In view of the above, this invention relates to a method for a prophylactic treatment of an HIV infection by administering toga patient as soon as possible after a suspected exposure to HIV a pharmacologically effective amount of an antiviral substance showing a mean initial suppression of viral load by 1.4 log or more when given alone for 14 days, and which does not reduce the number of lymphocytes, granulocytes and macrophages by more than 10% as determined by differential blood count after 12 weeks of treatment, or a pharmacologically effective amount of a drug combination comprising at least one such antiviral agent. A drug combination is preferred to completely avoid an infection of the individual. However, even if an infection cannot be completely prevented, due to high inoculation amounts of virus and/or a delayed start of therapy, the treatment according to the invention before or during the seroconversion phase allows the host to establish a long-lasting, specific, virus-containing immune surveillance and prevents the primary deletion of HIV-specific T cells, which is believed to be responsible for the state of T helper cell tolerance in chronic HIV infection.

In a further aspect, the invention relates to the use of an antiviral substance showing, when given alone for 14 days, a mean initial suppression of viral load by 1.4 log or more and which does not reduce the number of lymphocytes, granulocytes and macrophages by more than 10% as determined by differential blood count after 12 weeks of treatment, for the manufacture of a medicament for the immune system-assisted post-exposure prophylaxis of an HIV infection.

Moreover, a further aspect of the present invention is a prophylactic agent against HIV infection comprising an antiviral substance showing, when given alone for 14 days, a mean initial suppression of viral load by 1.4 log or more and which does not reduce the number of lymphocytes, granulocytes and macrophages by more than 10% as determined by differential blood count after 12 weeks of treatment. When administered post exposure, this agent serves to provide an efficient immune-system assisted prophylaxis against an HIV infection.

In yet a further aspect of this invention, an emergency pharmaceutical preparation kit for the post exposure prophylaxis of an HIV infection is provided, which comprises (a) a non-nucleosidic reverse transcriptase inhibitor of formula (I) as defined in claim 2 or a pharmaceutically acceptable derivative thereof, (b) a nucleosidic reverse transcriptase inhibitor, and (c) a protease inhibitor. In a preferred embodiment, this combination of antiviral agents consists of (3S)-ethyl-6-fluoro-4-isopropyloxycarbonyl-3,4-dihydroquinoxalin-2(1H)-one, lamivudine and neffinavir.

### BRIEF DESCRIPTION OF THE DRAWINGS

FIG. 1 schematically shows the course of a HIV viremia and of HIV antibodies in blood of three infected individuals A, B and C. It illustrates that different patients may have different heights/onsets of viremia and seroconversion as well as different setpoints of viremia. Setpoint is defined as the viremia after seroconversion.

FIG. 2 shows the results of a quantitative RNA PCR analysis of plasma samples from animals untreated and treated with Compound A. RT-SHIV levels in plasma are shown by the number of copies of viral RNA per ml.

FIG. 3 shows the humoral response against RT-SHIV in an ELISA analysis of plasma samples from untreated and Compound A-treated animals. 1:100 fold diluted plasma was assayed against SIVmac239 virion lysate. Antibody titer was monitored by the absorbance at 492 nm.

FIG. 4a shows the CTL activity against gag-pol protein of treated and untreated animals.

FIG. 4b shows the CTL activity against env protein of treated and untreated animals.

FIG. 4c shows the env specific proliferative response of treated and untreated animals.

### DETAILED DESCRIPTION OF THE INVENTION

The antiviral agent used in the present invention may be given either alone (monotherapy) or in combination with one or several other antiviral agents. Such further antiviral drugs are preferably selected from the group consisting of nucleosidic reverse transcriptase inhibitors, non-nucleosidic reverse transcriptase inhibitors and viral proteases. However, drugs with a different antiviral mechanism of action, e.g. via inhibition of the viral integrase, are likewise suitable. The antiviral drug or combination of drugs with the lowest potential for side effects and the highest suppression of viral load for each individual drug is preferred. Any drug or drug combination should not substantially (i.e. by more than 10%) lower the number of lymphocytes, granulocytes and macrophages as determined by differential blood count after 8, more preferably after 12 weeks of treatment, and preferably keep the number of these cells substantially (within 10%) constant after 12 weeks of treatment. This is what is meant by the expression “does not reduce the number of lymphocytes, granulocytes and macrophages by more than 10% after 12 weeks of treatment”. The number of lymphocytes, granulocytes and macrophages can be easily determined by a conventional differential blood count method.

The antiviral agent for use in the present invention must show a mean initial suppression of the HI-viral load in seroconverted individuals harbouring sensitive viruses against this drug by at least 1.4 log units when given alone. It should preferably show an initial suppression of viral load by 1.5 log units or more, most preferably by 1.6 log units or more.

The term “initial suppression” as used herein means the suppression of viral load achieved in an HIV-seroconverted individual, who is treatment-naive for the respective class of substances and harbours a sensitive virus for treatment periods of 7 days, more preferably 14 days. The initial suppression is expressed as the quotient of viral load one day before treatment and after 7 or 14 days of treatment, respectively, provided that the patient achieves drug trough levels in plasma which correspond to at least 3 times the IC₉₀ concentration for that virus. The viral load is measured by the commercially available Chiron bDNA assay in plasma samples. More details of this test are disclosed in Nature Medicine (1996) 625-629. A “sensitive virus” means an HIV isolate displaying an IC₅₀ value of up to a factor of 2 of the mean IC₅₀ value determined for field isolates of the respective subtype B (as defined according to the principles by Myers et al, Human Retroviruses and AIDS, 1997, Los Alamos National Laboratory) from untreated individuals against the respective drug. It should be noted that the invention is not restricted to subtype B HIV-1 strains. The definition used herein is only to define the patient population in which the initial suppression of viral load is to be determined. Analogous measurements can be performed in patients infected with other HIV-strains.

The terms IC₅₀ and IC₉₀ mean an inhibitory concentration of the virus at which 50% or 90%, respectively, of the viral replication in cell culture is inhibited.

The term viral load means the number of HIV genome equivalents which can be detected by quantitative measurement of viral nucleic acids per ml of plasma or serum using the bDNA kit from Chiron.

To determine a mean value for the initial suppression of viral load about 15 individuals, selected as indicated above, should be treated. The administration is preferably p.o., but other administration routes which achieve the above-defined free plasma concentration of the drugs are also acceptable.

Several conventionally used antiviral agents such as zidovudine show a mean initial suppression of 1 log or less. Zidovudine, for example, is reported to have a suppression (change in viremia) of 1 log after 7 days of treatment at 1000 mg per day (Loveday et al, The Lancet 345, 820-24 (1995)). Such agents are less preferred for an efficient post-exposure prophylaxis according to the present invention but may still be used in combination with one or more other antiviral agents satisfying the above criteria, Nevertheless, it is preferred according to the invention that any drug combination only contains antiviral agents each of which; when given alone for 14 days, shows a mean initial suppression of viral load by 1.4 log or more and which does not reduce the number of lymphocytes, granulocytes and macrophages by more than 10% as determined by differential blood count after 12 weeks of treatment.

Preferred antiviral agents for use in the present invention, either alone or in combination, e.g. in a particularly preferred three-drug combination, are the NNRTI compounds described in EP-A-0 708 093 and the pharmaceutically acceptable derivatives thereof. They have the following formula (I):

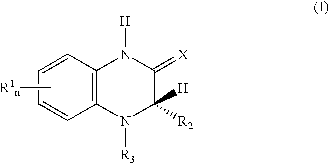

or a tautomeric structure of formula (Ia):

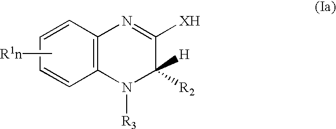

In the above formulae the substituents and symbols have the following meanings:

n=0, 1 or 2

R¹=F, Cl, HO, methoxy, ethoxy, n-propoxy, isopropoxy;

R²=C₁-C₄ alkyl, optionally substituted with OH, C₁-C₄ alkoxy, or C₁-C₄ alkylthio;

R3=C₁-C₆ alkyloxycarbonyl or C₂-C₆ alkenyloxycarbonyl

X=O, S or Se.

Particularly preferred is the use of a compound selected from (3S)-ethyl-6-fluoro-4-isopropyloxycarbonyl-3,4-dihydroquinoxalin-2(1H)-one and (3S)-6-methoxy-3-methylthiomethyl-4-isopropyloxycarbonyl-3,4-dihydroquinoxalin-2(1H)-thione and pharmaceutically acceptable derivatives thereof. (3S)-ethyl-6-fluoro-4-isopropyloxycarbonyl-3,4-dihydroquinoxalin-2(1H)-one having the formula

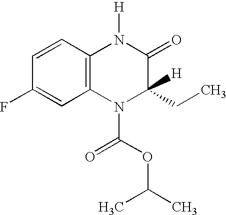

is most preferred. It has been found that this compound has surprisingly good human pharmacokinetic properties, high antiviral activity and a good tolerability combined with a low level of side-effects.

Pharmacologically acceptable derivatives of the compounds of formula (I) include the salts, hydrates, solvates and prodrugs of these compounds.

Compounds of formula (I) may be synthesized according to art-recognized methods. Reference may be made to EP-A-708 093 and DE-A-43 42 024, herein incorporated by reference, in this respect. These applications reference further literature on quinoxalines which may likewise be used for synthetic purposes, e.g. J. Liebigs Ann. Chem. 237, 327 (1986).

The term “post exposure prophylaxis” as used herein is not limited to an actual exposure to HIV but also includes a suspected exposure where it is unclear whether or not the virus has been transmitted. Thus, the present invention may likewise be applied after a suspected or likely HIV exposure when a detection of HIV in the treated subject is not (yet) possible.

A medicament containing the antiviral agent for use according to the present invention should be administered as soon as possible after exposure, and preferentially before a viremia of the retrovirus starts, which may be associated with signs of the acute retroviral syndrome. However, based on the present invention a PEP treatment may also be applied at times when the virus replication has started or is likely to have started in the exposed individual in order to reduce the peak or duration of the primary viremia, thus enabling the immune-system assisted control of the virus.

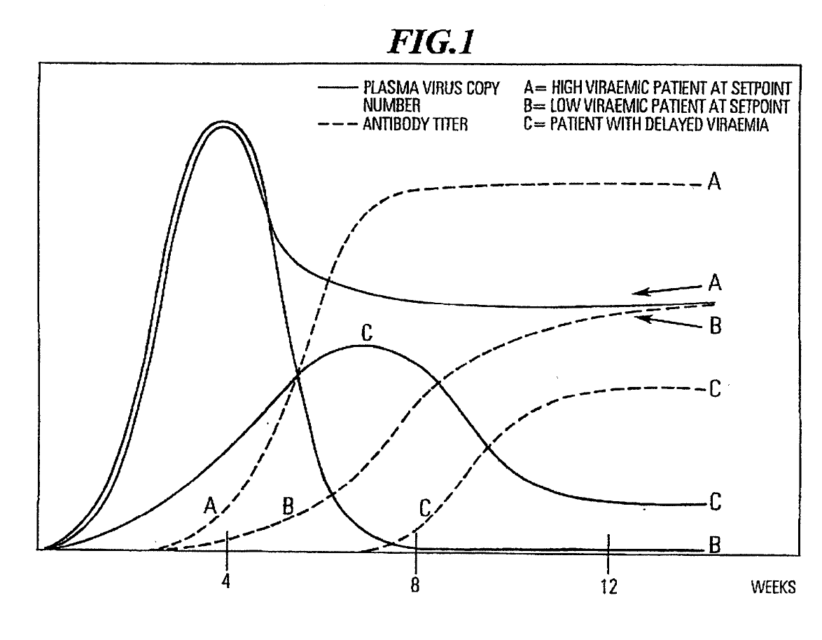

A preferable time window for post-exposure prophylaxis is about 0 to 24 hours, still more preferably 0 to 12 hours and most preferably 0 to 1 hours with the aim to prevent an infection from establishing. However, based on the present invention and in contrast to the current state of the art, a clinical benefit can also be expected when administration is started at later times, including during seroconversion. Seroconversion is defined as a state where (HIV) antibodies are detectable in a subject. Full seroconversion means the presence of all antibodies detected by Western Blots and used for confirmatory assays. As shown in FIG. 1, in most humans full seroconversion is observed after about 4-8 weeks post exposure (A, B) but in some, it may take up to 12 weeks (C). The clinical benefit of the present invention is based on the efficacy of the immune system assisted post-exposure prophylaxis, even if an infection cannot be prevented. Thus, one aspect of the present invention is the use of an antiviral agent as defined above for the preparation of a medicament for the immune-system assisted post exposure prophylaxis of an HIV infection wherein the treatment is started even after 72 h following exposure but before full development of the primary viremia. When the treatment is begun while seroconversion has already started, the likelihood to establish a long-term virus-containing immunity will be reduced. Such conditions are less preferred but are included in the present invention.

The administration should preferably be started before the primary viremia is fully developed. It is one important aspect of the present invention that a strong and well-tolerated antiviral agent is administered following HIV exposure so as to suppress the height and duration of primary viremia in the patient, even if the goal of preventing infection can no longer be achieved, In other words, the use of an antiviral substance which shows a mean initial suppression of HIV viral load by about 1.4 log or more for the preparation of a medicament for suppressing a primary HIV viremia constitutes a further aspect of this invention. As stated above, the medicament may be a combination of several drugs, preferably three antiviral agents, at least one of which satisfies the above requirement. The use of a compound of formula (I) and most preferably (3S)-ethyl-6-fluoro-4-isopropyloxycarbonyl-3,4-dihydroquinoxalin-2(1H)-one or a pharmaceutically acceptable derivative thereof, either alone or more preferably in combination with equally potent and well-tolerated drugs, for such application is most preferred.

The term “suppressing a primary HIV viremia” means that the treated individual develops a much less pronounced viremia compared with an untreated individual, i.e. that the first peak of the solid line in FIG. 1 is substantially lowered or even completely suppressed and that in cases where viremia is already present, its duration would be cut by the antiviral intervention.

The post-exposure prophylactic treatment according to the invention should be applied for at least 6 weeks, preferably for longer times, e.g. up to 12 weeks. This will, when there was an actual viral exposure, result in a long-lasting, specific immune surveillance which is sufficient to contain the viral load in a treated subject at a clinically acceptable level for prolonged periods of time, comparable to a long-term nonprogressor status. It is assumed that the immune response evoked by the method of the present invention id its most preferred embodiment is directed to more than one HIV antigen which enhances the efficiency of the immune control. Thus, the immune system assists in controlling the HIV load at a level which is clinically acceptable even after cessation of the treatment. The term “immune system assisted prophylaxis” should be construed accordingly.

The containment of the viral load in a treated subject at a clinically insignificant level will ideally last for the entire normally expected lifetime of the patient. As will be shown below, evidence obtained in monkeys (rhesus macaques) showed that this immune-system assisted control lasted for at least 40 weeks following exposure, in contrast to untreated animals. Indeed, no signs of a loss of viral suppression were observed in our studies during the entire period of observation.

Administration forms:

The medicaments for use in the present invention may be administered in any conventional way such as orally, parenterally (e.g. intravenously), rectally, subcutaneously, intramuscularly or topically. Oral administration is preferred. The most convenient administration mode is oral administration by tablets, capsules, lozenges or as a drink, given 1-3 times a day, most preferably once daily. Intravenous application or infusion may be preferred if a rapid onset of the antiviral action is desired, i.e. at times where prevention of the infection can still be expected. When a combination therapy comprising at least one antiviral agent which satisfies the requirements of the present invention is applied, this may be given as different compounds in one tablet or in the form of separate tablets.

A suitable daily dose may be in the order of 0.1 to 30 mg/kg of the antiviral agent of formula (I), preferably 0.2 to 5 mg/kg/day, most preferably 0.5 to 3.5 mg/kg in an adult human. Other antiretroviral agents satisfying the requirements of the present invention should be dosed accordingly, taking into account their pharmacological properties and their antiretroviral efficacy.

In yet a further aspect of this invention, an emergency pharmaceutical preparation kit for the post exposure prophylaxis of an HIV infection is provided to prevent or control a suspected HIV infection following exposure. This emergency kit comprises (a) a non-nucleosidic reverse transcriptase inhibitor of formula (I) as defined above or a pharmaceutically acceptable derivative thereof, (b) a nucleosidic reverse transcriptase inhibitor, and (c) a protease inhibitor. In a preferred embodiment, this kit consists of (3S)-ethyl-6-fluoro-4-isopropyloxycarbonyl-3,4-dihydroquinoxalin-2(1H)-one, lamivudine and nelfinavir. A particularly recommendable dosage for an adult human may be 2×150 mg/day of lamivudine, 3×750 mg/day of nelfinavir and 1×200 mg/day of (3S)-ethyl-6-fluoro-4-isopropyloxycarbonyl-3,4-dihydroquinoxalin-2(1H)-one.

Best Mode of the Invention

The use of (3S)-ethyl-6-fluoro-4-isopropyloxycarbonyl-3,4-dihydroquinoxalin-2(1H)-one for the post exposure prophylaxis of HIV-1 is presently considered to be the best mode of this invention. This compound is preferably administered in combination with at least one further antiretroviral agent such as preferably lamivudine which combines efficacy with good tolerability and few side effects. The treatment should start as soon as possible after exposure, ideally immediately, and be continued over about 12 weeks. A particularly preferred dosage regimen for an adult individual is a 200 mg once daily-tablet of (3S)-ethyl-6-fluoro-4-isopropyloxycarbonyl-3,4-dihydroquinoxalin-2(1H)-one. Only immunocompetent patients will benefit from the inventive immune-system assisted post-exposure prophylaxis.

The effects of the invention will be illustrated in the following experimental results:

### EXAMPLE

The following example shows the post-exposure prophylaxis with (3S)-ethyl-6-fluoro-4-isopropyloxycarbonyl-3,4-dihydroquinoxalin-2(1H)-one (hereinafter COMPOUND A) in rhesus monkeys.

Methods

Preparation of Virus RT-SHIV:

The virus used in the experiments is a chimeric simian-human immunodeficiency virus (SHIV) that consists of SIVmac239 virus genome with replacement of reverse transcriptase gene (RT) by the corresponding HIV-1 RT gene (Ueberla, K. et al (1995), Medical Sciences 92, 8210-5214). RT-SHIV induced AIDS in experimentally infected rhesus monkeys (Ueberla, loc.cit.). Proviral RT-SHIV DNA was prepared by ligation of RT-SHIV5′ and 3′ half of SIVmac239. COS-1 virus stocks were prepared by DNA transfection of proviral DNA. Virus stocks for efficacy experiments were prepared by propagation of COS-1 virus stock in rhesus monkey peripheral blood lymphocytes (PBL). P27 antigen concentration of the virus stocks was determined with a commercial SIV gag antigen ELISA kit (Coulter). The 50% T cell infective dose (TCID₅₀) of the stocks was determined with Herpes Simiri Virus transformed cynomolgus CD4⁺ T cells (cyT/HSV).

Inhibitory Activity of COMPOUND A on RT-SHIV Replication in Herpes Saimiri Virus Transformed Monkey CD4⁺ T Cells:

3×10⁶ of CyT/HSV cells were infected with RT-SHIV stocks containing 10000 TCID50 for 2 h at 37° C., then washed and resuspended in the medium containing serially diluted COMPOUND A (0, 0.1, 0.3, 1, 3, 10, 30, 100, 300, 1000 nM). Cells were kept in the culture condition by half medium exchange with medium containing 2 fold concentration of COMPOUND A every 2 days. Virus growth was monitored by measuring of P27 SIV gag antigen in culture supernatant with a commercial kit.

Animals and Study Plan:

Nine rhesus monkeys (male, 2 years old, 2-3 kg) were seronegative for SIV, Simian T cell Leukemia Virus (STLV), B virus and Type D retroviruses. 15 mg/kg body weight of COMPOUND A was subcutaneously administered into animals twice a day (12 h interval) for 28 d. Animals were divided into three groups. All animals were intravenously administered with 100 TCID₅₀ of RT-SHIV The first group consisted of three animals treated with COMPOUND A from 8 h post exposure for 28 d. The second group of animals was treated with COMPOUND A from 24 h post exposure for 28 d. The third group (control) received the solvent that was used to dissolve COMPOUND A (PEG400:glycerol:water=969:60:100) from 8 h post exposure for 28 d.

Sampling of Specimen:

Blood was collected from all the animals either just before or 2 h after the drug administration on 0, 3, 7, 10, 14, 21, 29 d post exposure in order to check the drug concentration, otherwise in intervals of 2 to 8 weeks. Biopsy of inguinal lymph nodes was conducted on 17 d or 18 d and 8 weeks post exposure in two animals per group. Cerebrospinal fluid (CSF) was collected from the animals just before biopsy.

Determination of COMPOUND A Concentration in Plasma and CSF:

Blood was collected from all animals just before or 2 h after drug administration to check trough (lowest) or highest blood concentration of the drug. COMPOUND A concentration in CSF was determined by HPLC.

Determination of Plasma Viral Load:

Viral RNA was purified from plasma with a commercial viral RNA isolation kit (Boehringer Mannheim). Gag cDNA was PCR amplified with a commercial RNA PCR kit (Boehringer Mannheim) with the gag primers SG05i (5′-ACTGCTGATTCAAAATGCiMCC-3′), SG06i (5′-CTACTGGTCTiCTCCAAAGAGAGAATTG-3′) according to the protocol by Piatak et al. (Biotechniques 14, 70-81-1993) with modifications. Amplified gag DNA was separated by 2% agarose electrophoresis and cyber green stained DNA was analyzed with a fluorescence image analyzer (FLA2000, Fuji Photo Film). Quantitative determination of the viral RNA copy number was calculated using external standards.

T-SHIV DNA Amount in Peripheral Blood Mononuclear Cells (PBMC) and Lymph Node Cells (LNC):

PBMC were separated by Ficoll-Hypaque gradient centrifugation. Cellular DNA was purified from 1×10⁶ cells with a commercial DNA purification kit (Qiagen). Gag sequence of viral DNA was PCR amplified with the primers SG05i and SG06i. Nested PCR amplification with two sets of primers, i.e. F34C (nt. 9065 to 9082)(5′-CCTACCTACAATATGGGT-3′) and F35C (nt. 9800 to 9778)(5′-CCTCTGACAGGCCTGACTTGCT-3′), the second primers: N3 (nt. 9182 to 9201)(5′-GMGATGGATACTCGCAATC-3′) and N4, (nt. 9552 to 9533)(5′-TMTCCTGCCAATCTGGTAT-3′) was performed to detect one copy per 100,000 cells. Detection and quantitative determination of the viral DNA copy number was conducted as described for plasma viral load. The cell numbers of PBMC and LNC were estimated by imaging analysis of cyber green-stained chromosomal DNA separated by agarose gel electrophoresis.

Anti-SIV ELISA:

1:100 fold diluted plasma was assayed with ELISA plates coated with SIVmac239 virion lysate. Antibody titer was monitored by measuring the absorbance at 492 nm.

Flow Cytometry:

Blood lymphocytes were stained with monoclonal antibodies against human CD4, CD8, CD16, CD20, CD 29 and monkey CD3 directed conjugated FITC or PE then analyzed with a Fluorescence Activated Cell Sorter (FACS) Cavalier and Cell Quest software (Becton & Dickinson).

Cytotoxic Lymphocytes (CTL) assay:

The method used was described previously (J. Virol. 1993: 67, 1707-1711). PBMC stored at −150° C. were thawed and incubated in RPMI medium for 3 days at 10⁶/ml with ConA (5 μg/ml), washed, and then maintained for another 3 days in medium supplemented with human IL2 (2 ng/ml) at 37° C. in a CO₂ incubator. Target cells, autologous Herpes Papio transformed B-LCL, were infected with recombinant vaccinia virus (rvv) expressing SIV proteins (SIVmac251-gag-pol or SIVmac239-env), or parental control vv, (ATCC VR-325) for 16 h at 37° C. in a CO₂ incubator. Target cells: B-LCL infected with vv were labeled with ⁵¹Cr, then incubated with effector cells for 5 h. Specific lysis was calculated as % SIV env or gag-pol specific lysis-% of control vv infected target cells.

Cell Proliferation Assay:

The method used was described previously [J. Virol, 1996: 70, 678-681]. In brief, PBMC was thawed and incubated as described in the CTL assay. Triplicates of PBMC (4×10⁵) cells were incubated with autologous B-LCL (1×10⁵) that had been infected with SIV env-rvv or control vv and fixed with 1.5% paraformaldehyde in phosphate buffered saline (PBS). ³H-thymidine was added to 8 h before harvesting. The incorporation of ³H-thymidine was measured by liquid scintillation counting. The stimulation index was calculated as (mean c.p.m. of PBMC cultured with env-rvv/mean c.p.m. of PBMC cultured with control vv).

Results

Antiviral Activity and Pharmacokinetics of COMPOUND A:

The inhibitory activity of COMPOUND A on RT-SHIV replication was studied with Herpes Saimiri Virus transformed monkey CD4⁺ cells. As 10 nM COMPOUND A suppressed more than 95% of RT-SHIV replication, the IC90 value was calculated to be 8 nM. Preliminary pharmacokinetics studies in rhesus monkeys showed that subcutaneous administration of the compound; 15 mg/kg weight b.i.d. in 12 h intervals resulted in a trough concentration of more than 100 ng/ml which is 3 fold higher than the IC90 value when the protein binding ratio is taken into consideration. Four weeks of drug administration maintained the minimum level of the drug higher than the effective minimal concentration without any adverse effect in all animals. The concentration in cerebral spinal fluid (CSF) was approximately one tenth of that in blood.

RT-SHIV replication in untreated animals:

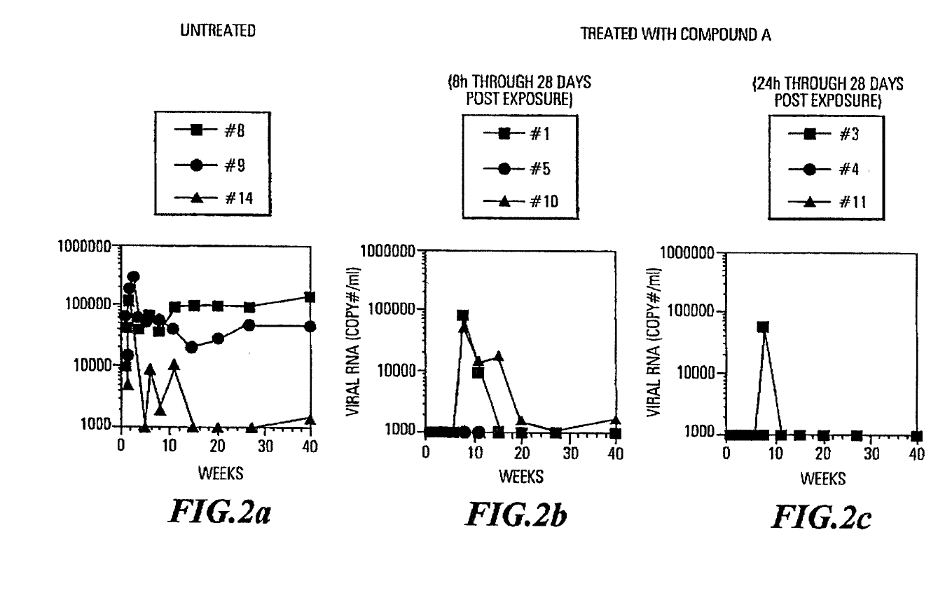

Viral RNA was detected from 7 or 10 d post exposure up to 40 weeks post exposure in all plasma specimen from two animals (FIG. 2). In all three animals a viremia peak at 1-3×10⁵ RNA copy/ml was observed at 2 or 3 weeks post exposure. While in two animals plasma viral RNA levels persisted around 10⁵ copy/ml after a moderate reduction occurred at 4 weeks post exposure, viral RNA levels in one animal declined to about 10³ copy/ml after 15 weeks post exposure (FIG. 2). The RT-SHIV infection in PIBMC was monitored by quantitative virus isolation (QVI) and quantitative PCR assay of proviral DNA. Serially diluted PBMC were co-cultured with C8166 to determine the minimal PBMC cell number to provoke a productive infection of C8166 cells. The number from the QVI test indicates infectious cells per one million PBMC. QVI results were consistent with plasma viral RNA levels. The quantitative results are shown in Table 1. The numbers indicates the results of QVI. A + or − on the right indicates results of viral RNA PCR assay for the detection of virus in culture supernatant. +=positive, −=negative.

TABLE 1
Quantitative virus isolation (QVI) and PCR

A. Untreated

| | QVI/PCR | | | |
|---|---|---|---|---|
| weeks post exp. | #8 | #9 | #14 | (animal no.) |
| 2 | *16/+ | 16/+ | 16/+ | |
| 6 | 16/+ | 16/+ | 4/+ | |
| 8 | 64/+ | 16/+ | 1/+ | |
| 11 | 16/+ | 1/+ | −/− | |
| 15 | 4/+ | 1/+ | −/− | |

B. Treated with Compound A

| | 8h thru 28 d post exp | | | 24h thru 28 d post exp. | | |
|---|---|---|---|---|---|---|
| w post exp. | #1 | #5 | #10 | #3 | #4 | #11 |
| 2 | −/− | −/− | −/− | −/− | −/− | −/− |
| 6 | 4/+ | −/− | 1/+ | 16/+ | −/− | −/− |
| 8 | 16/+ | −/− | 16/+ | 16/+ | 1/+ | 4/+ |
| 11 | 4/+ | −/− | 4/+ | 16/+ | −/− | 4/+ |
| 15 | −/− | −/− | −/− | −/− | −/− | −/− |

Proviral DNA levels which measure a persistent viral burden rather than a viremia showed that some hundreds proviral DNA copies/100,000 cells in PBMC were detected in all the specimens assayed from the untreated animals from 2 w post exposure through 50 w post exposure. This is shown in Table 2. The upper block of Table 2 shows the no. of viral DNA copies/100,000 cells determined in PBMC at 2, 4, 15, 40, and 50 weeks post exposure. The lower line shows the corresponding number in LNMC at a weeks post exposure. The detection limit was 1 copy DNA per 100,000 cells. In contrast to the untreated animals, the proviral DNA levels were very low in the treated groups.

TABLE 2
RT-SHIV proviral DNA in PBMC and LNMC

| | | | | Treated | | | Treated | | |
|---|---|---|---|---|---|---|---|---|---|
| Untreated | | | | 8h thru 28 dpi | | | 24 h thru 28 d pi | | |
| w p.e. | #8 | #9 | #14 | #1 | #5 | #10 | #3 | #4 | #11 |
| 2 | 300 | 180 | 848 | 0 | 0 | 0 | 0 | 0 | 0 |
| 4 | 340 | 1594 | 236 | 0 | 0 | 0 | 0 | 0 | 0 |
| 15 | 150 | 74 | 417 | 1 | 0 | 7 | 16 | 0 | 1 |
| 40 | 633 | 273 | 25 | 0 | 0 | 1 | 0 | 0 | 0 |
| 50 | 241 | 339 | 31 | 0 | 0 | 5 | 2 | 0 | 0 |
| 8 | | 1773 | 307 | | 2 | | | 1 | |

COMPOUND A Suppressed RT-SHIV Replication in the Monkeys:

Analyses on plasma viral RNA, proviral DNA and infectious cells in PBMC demonstrated that viral replication was inhibited at a level below the sensitivity of the assays during the treatment and the following 3 weeks in all treated animals (FIG. 2). 4 weeks after cessation of treatment, a short active viral replication was observed in 50% of the animals (FIG. 2). In the rest of the animals, plasma viral RNA levels were kept lower than the detection level. QVI showed that another two animals had a few infectious cells in PBMC by 8 or 11 weeks post exposure (Table 2). In animal #5 no viral infection was observed by any of these methods.

Subsequent to the active viral replication around 10 weeks post exposure, viral replication was suppressed near or lower than the RNA PCR detection level (1000 copy/ml) and kept at this level up to 40 weeks post exposure (FIG. 2). Proviral DNA levels confirmed that a far lower level of persistent infection occurred in the treatment group relative to the untreated group Table 1).

Humoral Responses Against RT-SHIV:

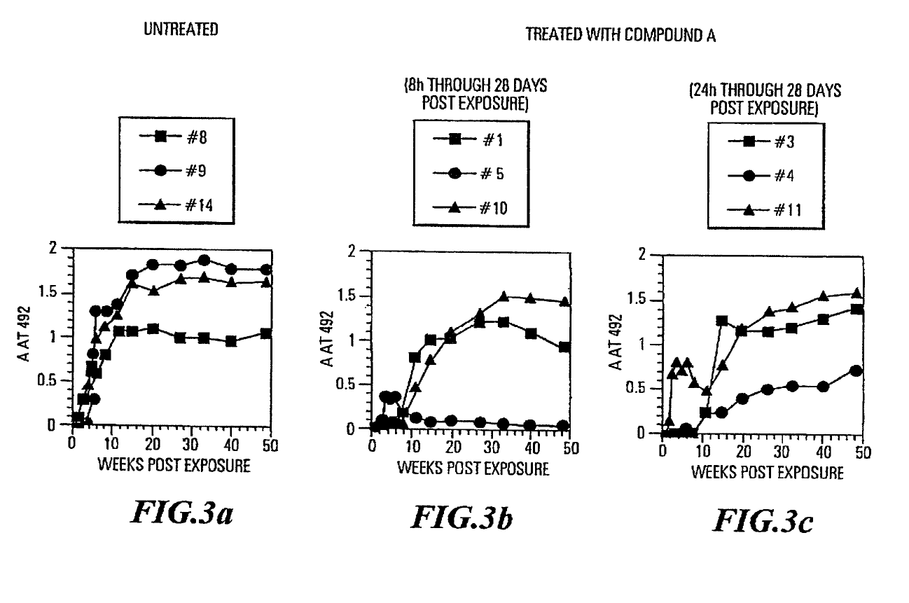

Humoral response against RT-SHIV was measured by ELISA against SIV virion proteins. Untreated animals became sero-positive at 4 weeks post exposure, thereafter a high antibody titer was sustained (FIG. 3). COMPOUND A treatment delayed induction of humoral response. Two of six treated animals became sero-positive at 11 weeks post exp., i.e. 5 weeks after cessation of the treatment. Although the rest of the animals eventually became sero-positive as the untreated animals did, antibody titer dropped later. One animal, #5, eventually lost its humoral response against the virus (FIG. 3).

Cell Mediated Immune Responses Against RT-SHIV:

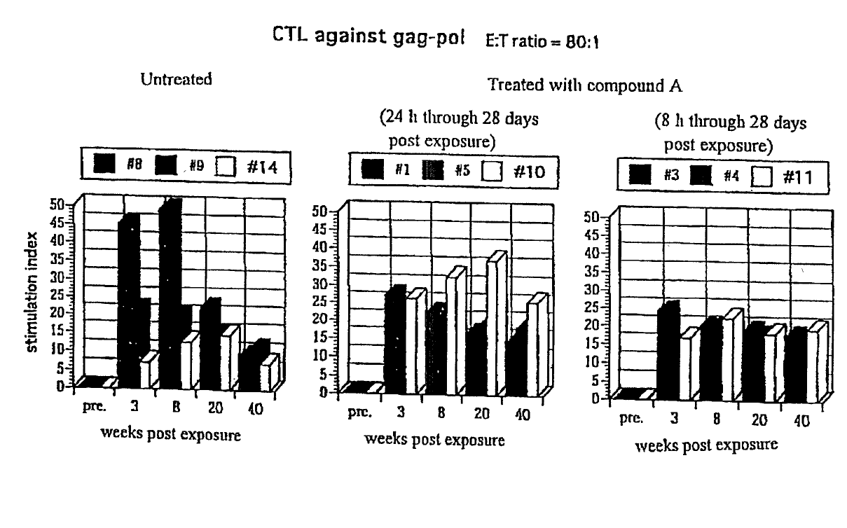

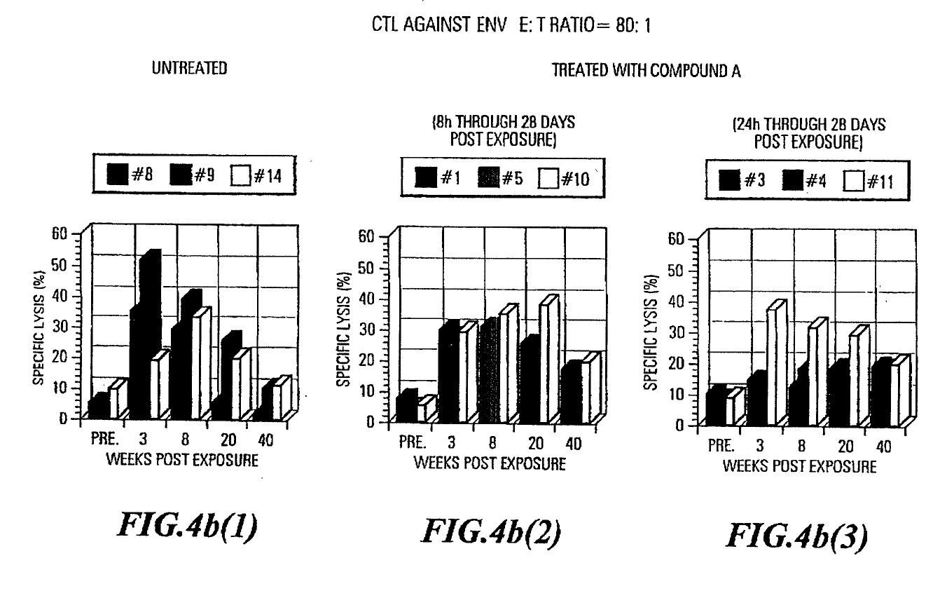

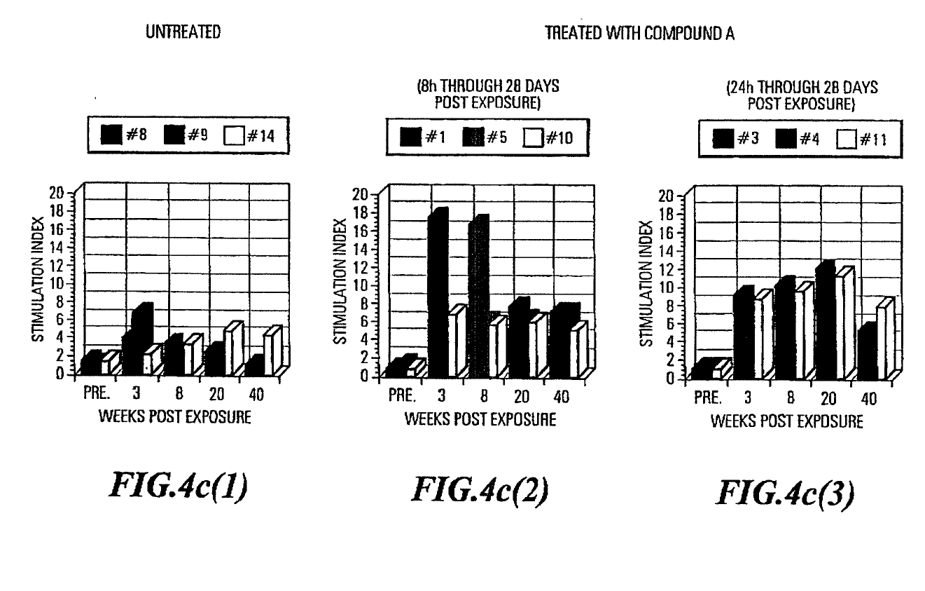

The virus specific cell mediated immune responses were studied by measuring CTL against env and gag-pol, and by env specific proliferation assays. Peripheral blood mono-nuclear cells at 6 d before infection, 3 w, 8 w, 20 w, and 40 w post exposure were subjected to CTL and proliferation assays. In untreated animals, high CTL activity against viral proteins was detected at 3 w and 8 w pi, but decreased at 20 w and 40 w post exposure in the untreated animals #8 and #9. In the other untreated animal, #14, delayed and sustained CTL activities were observed and correlated with a contained viral load at 15 w post exposure and thereafter (FIGS. 4a and 4b). In treated animals, a constant high CTL activity was detected through all time points tested (FIGS. 4a and 4b).

HIV-specific CD4⁺ helper activity measured as cell proliferative response to viral antigens is maintained in HIV infected long term non-progressors but not in disease progressors. Significantly higher env specific proliferative responses were observed as early as 3 weeks post exposure and continued through 40 weeks post exposure in all treated animals (FIG. 4c).

In contrast, the highest env-specific proliferative response was detected at 3 weeks post exposure in untreated animals and it was lower than in treated animals. Most importantly, it declined thereafter, except for animal #14 which had shown the lowest viral load in primary viremia.

Significanly, all treated animals developed a proliferative response and kept it at levels higher than those of the untreated group at week 40.(FIG. 4c).

Further aspects of the present invention include the following:

1. A method for the immune system-assisted post-exposure prophylaxis of an HIV infection, comprising administering to a subject a pharmacologically effective amount of an antiviral substance which, when given alone for 14 days, shows a mean initial suppression of viral load by 1.4 log or more and which does not reduce the number of lymphocytes, granulocytes and macrophages by more than 10% as determined by differential blood count after 12 weeks of treatment.

2. A method for the immune system-assisted post-exposure prophylaxis of an HIV infection, comprising administering to a subject a pharmacologically effective amount of an antiviral substance which is a non-nucleosidic reverse transcriptase inhibitor of formula (I) or a tautomer or a pharmacologically acceptable derivative thereof:

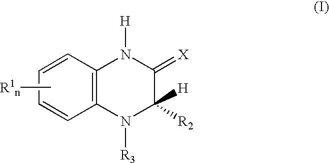

wherein the substituents and symbols have the following meanings:

n=0, 1 or 2

R¹=F, Cl, HO, methoxy, ethoxy, n-propoxy, isopropoxy;

R²=C₁-C₄ alkyl, optionally substituted with OH, C₁-C₄ alkoxy, or C₁-C₄ alkylthio;

R3=C₁-C₆ alkyloxycarbonyl or C₂-C₆ alkenyloxycarbonyl

X=O, S or Se.

3. The method according to aspect 2 wherein the compound of formula (I) is selected from (3S)-ethyl-6-fluoro-4-isopropyloxycarbonyl-3,4-dihydroquinoxalin-2(1H)-one and (3S)-6-methoxy-3-methylthiomethyl-4-isopropyloxycarbonyl-3,4-dihydroquinoxalin-2(1H)-thione and pharmaceutically acceptable derivatives thereof.

4. The method according to any of aspects 1 to 3 wherein the said antiviral substance of formula (I) is administered in combination with one or more further antiviral substances.

5. The method according to any preceding aspect, wherein the post-exposure prophylactic treatment is started as soon as possible after a (suspected) exposure to HIV and before the completion of a primary viremia and seroconversion.

6. The method according to any preceding aspect, wherein the post-exposure prophylaxis is terminated after 12 weeks.

7. The method according to any preceding aspect, wherein the post exposure prophylactic treatment establishes a specific immune surveillance sufficient to contain the viral load in a treated subject at a clinically insignificant level.

8. The method of an antiviral substance or substance combination as defined in any of aspects 1-4 for the manufacture of a medicament against a suspected HIV infection.

9. The method of an antiviral substance or substance combination as defined in any of aspects 1-4 for the manufacture of a medicament for an immune system-assisted post exposure prophylaxis by suppressing a primary HIV viremia.

10. The method according to any preceding aspect wherein the treatment is started after 72 h following exposure but before completion of primary viremia and seroconversion.

11. The method according to any preceding aspect, wherein the medicament is suited for once daily administration.

1. The use of an antiviral substance which, when given alone for 14 days, shows a mean initial suppression of viral load by 1.4 log or more and which does not reduce the number of lymphocytes, granulocytes and macrophages as determined by differential blood count after 12 weeks of treatment
for the manufacture of a medicament for the immune system-assisted post-exposure prophylaxis of an HIV Infection.

2. The use of an antiviral substance, which is a non-nucleosidic reverse transcriptase inhibitor of formula (I) or a tautomer thereof or a pharmacologically acceptable derivative thereof: 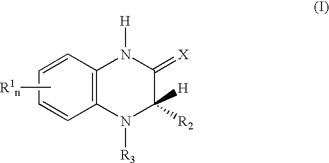
wherein the substituents and symbols have the following meanings:
n=0, 1 or 2
R¹=F, Cl, HO, methoxy, ethoxy, n-propoxy, isopropoxy;
R²=C₁-C₄ alkyl, optionally substituted with OH, C₁-C₄ alkoxy, or C₁-C₄ alkylthio;
R³=C₁-C₆ alkyloxycarbonyl or C₂-C₆ alkenyloxycarbonyl
X=O, S or Se.
for the manufacture of a medicament for the immune system-assisted post-exposure prophylaxis of an HIV infection.

3. The use according to claim 2 wherein the compound of formula (I) is selected from (3S)-ethyl-6-fluoro-4-isopropyloxycarbonyl-3,4-dihydroquinoxalin-2(1H)-one and (3S)-6-methoxy-3-methylthiomethyl-4-isopropyloxycarbonyl-3,4-dihydroquinoxalin-2(1H)-thione and pharmaceutically acceptable derivatives thereof.

4. The use according to any of claims 1 to 3 wherein the said antiviral substance is used in combination with one or more further antiviral substances.

5. The use according to any preceding claim, wherein the post-exposure prophylaxis is started as soon as possible after a (suspected) exposure to HIV and before the complete development of a primary viremia and seroconversion.

6. The use according to any preceding claim, wherein the post-exposure prophylaxis is terminated after 12 weeks of start of therapy.

7. The use according to any preceding claim, wherein the post exposure prophylactic treatment establishes a specific immune surveillance sufficient to contain the viral load in a treated subject at a clinically acceptable level.

8. The use of an antiviral substance or substance combination as defined in any of claims 1-4, for the manufacture of a medicament against a suspected HIV infection.

9. The use of an antiviral substance or substance combination as defined in any of claims 1-4 for the manufacture of a medicament for the immune-system assisted post-exposure prophylaxis of an HIV infection by suppressing a primary HIV viremia.

10. The use according to any preceding claim wherein the treatment is started after 72 h following exposure but before full development of a primary viremia and seroconversion.

11. The use according to any preceding claim, wherein the medicament is suited for once daily administration.

12. An emergency pharmaceutical preparation kit for preventing or controlling an HIV infection following a suspected exposure to HIV, which comprises (a) a compound of formula (I) as defined in claim 2 or a pharmaceutically acceptable derivative thereof, (b) a nucleosidic reverse transcriptase inhibitor, and (c) a protease inhibitor.

13. The emergency pharmaceutical preparation kit of claim 12 wherein the compound of formula (I) is (3S)-ethyl-6-fluoro-4-isopropyloxycarbonyl-3,4-dihydroquinoxalin-2(1H)-one.

14. The emergency pharmaceutical preparation kit of claim 12 or 13 wherein the protease inhibitor is nelfinavir.

15. The emergency pharmaceutical preparation kit of any of claims 12-14 wherein the nucleosidic reverse transcriptase inhibitor is lamivudine.
